(19) United States
(12) Patent Application Publication
(10) Pub. No.: US 2004/0005280 A1
(43) Pub. Date: Jan. 8, 2004

(54) Nail cosmetic compositions

(75) Inventor: Ginette Godbout, Ville St-Laurent (CA)

Correspondence Address: OBLON, SPIVAK, MCCLELLAND, MAIER & NEUSTADT, P.C., 1940 DUKE STREET, ALEXANDRIA, VA, 22314, US

(73) Assignee: FORMULE DIAMANCEL, INC., Ville Saint-Laurent (CA)

(21) Appl. No.: 10/435,200

(22) Filed: May 12, 2003

Related U.S. Application Data
(60) Provisional application No. 60/388,368, filed on Jun. 14, 2002.

(30) Foreign Application Priority Data
May 10, 2002 (CA) 2,383,296

(51) Int. Cl. A61K 7/04

(52) U.S. Cl. 424/61

(57) ABSTRACT

The invention relates to a nail cosmetic composition comprising a source of calcium in powder form and a cosmetically acceptable carrier therefor, the source of calcium being selected from the group consisting of pearl, ivory, seashell and mixtures thereof. The invention further relates to a nail enamel composition comprising a film former, an organic solvent, and a source of calcium in powder form, wherein the source of calcium is selected from the group consisting of pearl, ivory, seashell and mixtures thereof. Methods for applying the compositions of the invention are also disclosed.

### TECHNICAL FIELD

The present invention relates to improvements in the field of nail care products. In particular, this invention relates to cosmetic compositions for improving the structure of nails.

### BACKGROUND OF THE INVENTION

The nails of mammals, particularly the fingernails and toenails of humans, are composed of epidermis transparent cells called corneocyte cells, which are produced at the root of the nail. These cells join together to form a solid and continuous surface on the back of the terminal phalanges.

Each nail adheres intimately to the underlying support that forms the bed of the nail. The visible part of the nail is called the body of the nail. The hidden part is called the root.

The propagation of the corneocyte cells in the root ensures nail growth. The new cells go through a transformation in the solid layers and dry until they form a solid plate. The nail permanently receives continuous additions of cells at the level of its bed, which push the bed beyond the extremity of the phalange.

Keratin fibrils present inside the corneocyte cells, as well as the intercellular cements present, bring rigidity and flexibility to the nails.

Unfortunately, the fragility and vulnerability of the nails are observed in presence of mechanical, thermal and/or chemical attacks. For example, nails which are brittle and friable eventually break, split or crack due to mechanical shocks. The nails may also be degraded in the presence of aggressive chemical agents, particularly those present in household products. Nails degradation may also be a secondary effect of the ageing process. Degradation of nails can be very painful, unpleasant and unsightly, and permit to foreign bodies to cause infection to the nails.

Numerous approaches have been investigated in order to improve the resistance of the nails to different aggressions of mechanical, thermal, chemical or temporal nature by hardening or strengthening them. Various compositions based on chemical products such as synthetic gums of an acrylic polymer film, varnishes based on nitrocellulose combined with inert glass fibers and other hardening agents have been proposed respectively in U.S. Pat. Nos. 5,508,027, 4,873,077, 4,482,538 and 5,785,959. Although these compositions may have an hardening or strengthening action on the nails, they do not allow one to improve the structure of the nails. If any one of these compositions is applied to fragile or soft nails, harder nails may be obtained, but the latter will still have a tendency to crack and break.

Several approaches have also been investigated in order to improve the flexibility and resistance of the nails by nourishing and moisturizing them. Many attempts have been made to prevent the nails from dehydrating and to protect them against various types of aggressions. Compositions comprising synthetic active agents such as moisturizing, antimicrobial or antifungal agents have been extensively proposed.

U.S. Pat. No. 6,517,863 discloses compositions and methods for improving durability and hardness of the nail body. The compositions increase the durability of the nails by forming a protective layer of hydroxyapatite or other calcium phosphate mineral on the nail. Such compositions includes synthetic and inorganic bioactive glasses as active agents. These inorganic glass materials are bound to the nails and comprise a plurality of synthetic chemicals products.

U.S. Pat. No. 5,645,823 discloses an aqueous solution for restoring and maintaining the compositional balance of normal, healthy keratinous tissue and their adjacent structures. The latter composition comprises a plurality of synthetic compounds used as antimicrobial or active agents

U.S. Pat. No. 4,919,920 discloses compositions and methods for reinforcing and hardening mammals keratinized appendages, wherein fluoride ions in an aqueous cosmetic carrier are topically applied. The utilization of fluoride ions increases both the hardness and the resistance of the nail. However, this method presents the drawback of generating in situ acidity. U.S. Pat. No. 6,478,551 also discloses the use of fluoride ions for strengthening nails. A non-aqueous organic composition containing ammonium hexafluorophosphate is used to provide an effective amount of fluoride ions to the nail body. In the latter two cases, harmful acidic by-products can potentially be formed.

Although the above-mentioned compositions may prevent brittleness of the nails, they all comprise synthetic chemicals as main active agents, in respect of which long term and/or secondary effects are still not completely known.

During the last decade, in the field of pharmaceutical products, nutrition and cosmetic cares, the attitude of our society in respect of synthetic products has considerably changed. The trend has been to avoid synthetic products as much as possible and replace them by natural or organic products. The emergence of functional food, neutraceuticals and natural cosmetic products represents a good example of this trend. People are now more aware of the fact that frequent or long term consumption or utilization of certain chemicals may have negative and side effects on human health. Since many of these chemicals are quite recent, the long term effects are often unknown. Thus, natural or organic products have been extensively used as active agents in pharmaceutical or cosmetic products. For example, U.S. Pat. No. 5,667,768 discloses nail care compositions comprising an organic solution or dispersion of at least one natural active agent. However, the use of such compositions is tedious since they must be applied 2 to 3 times per week for at least 8 weeks in order to provide encouraging results.

### SUMMARY OF THE INVENTION

It is therefore an object of the present invention to overcome the above-mentioned drawbacks and to provide compositions for improving the structure of nails, which comprise as main active ingredient a natural active agent.

According to one aspect of the invention, there is provided a nail cosmetic composition comprising a source of calcium in powder form and a cosmetically acceptable carrier therefor, the source of calcium being selected from the group consisting of pearl, ivory, seashell and mixtures thereof.

According to another aspect of the invention, there is provided in a nail cosmetic composition comprising an active agent and a cosmetically acceptable carrier therefor, the improvement wherein the active agent is selected from the group consisting of pearl powder, ivory powder, seashell powder and mixtures thereof.

According to still another aspect of the invention, there is provided a nail enamel composition comprising:

a film former;

an organic solvent; and

a source of calcium in powder form, the source of calcium being selected from the group consisting of pearl, ivory, seashell and mixtures thereof.

According to yet another aspect of the invention, there is provided in a nail enamel composition comprising an active agent, a film former and an organic solvent, the improvement wherein the active agent is selected from the group consisting of pearl powder, ivory powder, seashell powder and mixtures thereof.

According to a further aspect of the invention, there is provided a method of treating nails, comprising the step of applying to the nails a nail cosmetic composition or a nail enamel composition according to the invention.

According to still a further aspect of the invention, there is provided a method of treating nails, comprising the step of applying to the nails at least one layer of a nail cosmetic composition or a nail enamel composition according to the invention.

According to yet a further aspect of the invention, there is provided an active agent for improving structure of nails, comprising a source of calcium in powder form, the source of calcium being selected from the group consisting of pearl, ivory, seashell and mixtures thereof.

Applicant has found quite surprisingly that by applying to the nails a nail cosmetic composition or a nail enamel composition as defined above, the treated nails have an improved structure. Nails treated with the compositions of the present invention are strengthened, and the risks of breaking, splitting and cracking are thus considerably reduced. Moreover, the treated nails have a healthy appearance and their growth rate is increased.

Even if the compositions of the invention can potentially contain few synthetic products, the main active agents used, such as pearl powder, ivory powder or seashell powder, are natural and mild, and are thus not detrimental to the users health.

The expression “a source of calcium” as used herein refers to pearl, ivory and seashell. Pearl and seashell comprise calcium carbonate and ivory comprises calcium phosphate.

The term “ivory” as used herein refers to ivory of an animal source. Preferably the animal is an elephant, mammoth, walrus, warthog, hippo, sperm whale or narwhale. More preferably, the animal is a mammoth or elephant.

The expression “pearl powder” as used herein refers to a natural pearl powder which is a source of calcium and more particularly calcium carbonate. The pearl powder is obtained by grinding a pearl originally contained inside the shell of a mollusk.

The expression “seashell powder” as used herein refers to a powder obtained by grinding the shell of mollusks. Such a powder preferably comprises nacre, which is also referred to as mother of pearl, amber poria pearl, concha margarita, marine calcium, or concha margaritaferae. Nacre, a highly ordered microlaminate composite of crystals and biopolymers, is found in the iridescent inner layer of mollusk shells. Its biomaterials comprise calcium carbonate in a matrix of proteins and glycoproteins.

The expression “suspending agent” as used herein refers to a thixotropic agent or a rheological agent.

The terms “¼ sec” and “½ sec” as used herein in respect of nitrocellulose viscosity refers to the necessary time for a ball to fall to a given depth in nitrocellulose. The percentage of nitrocellulose optionally present in the compositions of the invention is given on a dry basis.

### DESCRIPTION OF PREFERRED EMBODIMENTS

The source of calcium in powder form used in the compositions of the invention can be present in an amount of at least 0.5% by weight, based on the total weight of the composition. Preferably, the amount of calcium source is comprised between 1.0 and 30.0% by weight, and more preferably between 2.0 and 10.0% by weight. Use is preferably made of pearl powder.

In the nail cosmetic compositions of the invention, the cosmetically acceptable carrier can be present in an amount of 20 to 97% by weight, based on the total weight of the composition. Preferably, the amount of cosmetically acceptable carrier is comprised between 75 and 95% by weight. The cosmetically acceptable carrier can be selected from the group consisting of a cream, a foam, a gel, a solution, a nail varnish composition, a nail enamel composition, a nail glue, a nail resin and a spray. Preferably, the cosmetically acceptable carrier is a cream, a gel or a solution. The cosmetically acceptable carrier can also be selected from the group consisting of a nail enamel composition, a nail glue, a nail varnish composition and a nail resin. The source of calcium in powder form is preferably suspended in the cosmetically acceptable carrier.

In the nail enamel compositions of the invention, the film former can be present in an amount of 10 to 60% by weight, based on the total weight of the composition. Preferably, the amount of film former is comprised between 15 and 40% by weight, and more preferably between 20 and 30% by weight. Examples of film formers include but are not limited to acrylate esters, acrylate polymers, acrylate copolymers, acrylate terpolymers, cellulose acetates, cellulose acetate butyrate, cellulose acetate propionate, ethyl cellulose, methacrylate esters, methacrylate polymers, methacrylate copolymers, methacrylate terpolymers, nitrocellulose, polycarbonates, polyester resins, polyurethanes, polyvinylbutyral, polyvinyl acetates, polyvinyl acetate phthalates, a tosylamide epoxy resin, a tosylamide formaldehyde resin and mixtures thereof. Preferably, the film former is selected from the group consisting of nitrocellulose, a tosylamide epoxy resin, a tosylamide formaldehyde resin and mixtures thereof.

Alternatively, the film former can comprise a primary film former and a secondary resin. The primary film former can be selected from the group consisting of acrylate esters, acrylate polymers, acrylate copolymers, acrylate terpolymers, cellulose acetates, cellulose acetate butyrate, cellulose acetate propionate, ethyl cellulose, methacrylate esters, methacrylate polymers, methacrylate copolymers, methacrylate terpolymers, nitrocellulose and mixtures thereof. Preferably, the primary film former is nitrocellulose. The secondary resin can be selected from the group consisting of polyester resins, polyvinylbutyral, polyvinyl acetates, polyvinyl acetate phthalates, a tosylamide epoxy resin, a tosylamide formaldehyde resin and mixtures thereof. Preferably, the secondary resin is selected from the group consisting of a tosylamide epoxy resin, a tosylamide formaldehyde resin and a mixture thereof.

In the nail enamel compositions of the invention, the organic solvent can be present in an amount of 40 to 90% by weight, preferably 45 to 80% by weight and more preferably 50 to 75% by weight, based on the total weight of the composition. Examples of organic solvents include but are not limited to abietyl alcohol, acetone, benzyl alcohol, butanol, t-butanol, butoxyethanol, butoxypropanol, n-butyl acetate, sec-butyl acetate, 1,4-butylene, butylene glycol, cyclopentane, cyclohexane, diethylene glycol, diethylene glycol monomethyl ether, diethyl ketone, dihydroxyacetone, ethanol, ethyl acetate, glycol, heptane, hexane, isobutyl acetate, isopropyl acetate, methanol, methoxy ethoxy ethanol, methyl ethyl ketone, methyl acetate, methyl isobutyl ketone, propan-1-ol, propan-2-ol, n-propyl acetate, pentane, propylene glycol, propylene glycol monomethyl ether, toluene and mixtures thereof. Preferably, the organic solvent is n-butyl acetate, ethyl acetate or a mixture thereof.

In the nail enamel compositions of the invention, the source of calcium in powder form can be suspended in a mixture of the film former and the organic solvent.

The nail enamel compositions of the invention can further comprise a plasticizer. The plasticizer can be present in an amount of 1 to 25% by weight, based on the total weight of the composition. Preferably, the amount of plasticizer is comprised between 2 and 20% by weight, and more preferably between 5 and 15% by weight. Examples of plasticizers include but are not limited to benzyl benzoate, butyl acetylricinoleate, butyl glycolate, butyl stearate, camphor, citrate esters, diamyl phthalate, dibutyl phthalate, dibutyl tartrate, dioctyl phthalate, di-2-ethylhexyl phthalate, diisononyl phthalate, di-2ethylhexyl adipate, diisononyl adipate, dimethoxyethyl phthalate, glycerol triacetate, glyceryl acetylricinoleate, methoxypolyethylene glycol, octyl palmitate, polyethylene glycol, propylene carbonate, propylene glycol, tributoxyethyl phosphate, tributyl phosphate, tricresyl phosphate, trioctyl trimellitate, triphenyl phosphate and mixtures thereof. Preferably, the plasticizer is dibutyl phtalate.

When the nail enamel composition of the present invention comprises a plasticizer, the source of calcium in powder form can be suspended in a mixture of the film former, the organic solvent and the plasticizer.

The nail enamel compositions of the invention can further comprise a suspending agent. The suspending agent can be present in an amount of 0.10 to 15.00% by weight, based on the total weight of the composition. Preferably, the amount of the suspending agent is comprised between 0.25 and 10.00% by weight, and more preferably between 0.50 and 5.00% by weight. The suspending agent can be selected from the group consisting of attapulgite, bentonite, hectorite and mixtures thereof. Preferably, the suspending agent is bentonite and more preferably stearalkonium bentonite.

The compositions of the invention can also comprise an additive such antibacterial agents, antifungal agents, botanical extracts, fragrances, moisturizers, preservatives, UV filters, proteins, stabilizers, vitamins or mixtures thereof.

The active agent and composition according to the invention can be used for preventing friability of nails, improving the structure of nails, providing an increased growth rate of nails and/or increasing the thickness of nails.

The compositions of the invention are preferably capable of protecting, hardening and/or embellishing the nails. The methods of the invention are preferably efficient for protecting, hardening or embellishing the nails.

In the method for treating nails using a nail cosmetic composition according to the invention, the composition can be applied to the nails at least once a week for a period of at least 2 weeks. Preferably, the composition is applied to the nails at least once a week for a period of at least 3 weeks and more preferably for a period of at least 5 weeks.

In the method for treating nails which consist of applying at least one layer of a nail enamel composition according to the invention, the composition can be applied to the nails at least once a week for a period of at least 2 weeks. Preferably, the composition is applied to the nails at least once a week for a period of at least 3 weeks and more preferably for a period of at least 5 weeks. Preferably, prior to apply a new layer of the nail enamel composition, the previously applied layer is removed by applying to the nails a nail polish remover.

The nail cosmetic compositions and nail enamel compositions of the present invention can be prepared by simply mixing together the cosmetic ingredients described hereinabove by stirring. The cosmetic ingredients are added one by one to form the desired composition and, after each addition, the mixture is stirred so as to be homogenized. Examples of satisfactory equipment and how to use it are readily apparent to one of ordinary skill in the art of nail care products.

The present invention will be more readily understood with reference to the following non-limiting examples.

### EXAMPLE 1

| Cosmetic ingredients | Wt % |
|---|---|
| ¼ sec. nitrocellulose | 8.5 |
| ½ sec. nitrocellulose | 8.5 |
| tosylamide/epoxy resin | 4.7 |
| tosylamide/formaldehyde | 4.7 |
| dibutyl phtalate | 7.6 |
| n-butyl acetate | 33.3 |
| ethyl acetate | 25.7 |
| stearalkonium bentonite | 1.0 |
| methanol 95% | 1.0 |
| pearl powder | 5.0 |
| TOTAL | 100 |

### EXAMPLE 2

| Cosmetic ingredients | Wt % |
|---|---|
| ¼ sec. nitrocellulose | 8.7 |
| ½ sec. nitrocellulose | 8.7 |
| tosylamide/epoxy resin | 4.8 |
| tosylamide/formaldehyde | 4.8 |
| dibutyl phtalate | 7.7 |
| n-butyl acetate | 34.0 |
| ethyl acetate | 26.2 |
| pearl powder | 5.1 |
| TOTAL | 100 |

The nail enamel compositions of Examples 1 and 2 were tested so as to determine their efficacy for improving the structure of nails. In order to perform such a test, four female test subjects were selected. Firstly, the quality of the nails of the test subjects was quantified by using the 1 to 10 evaluation scale defined as follows:

grades 1 to 3 correspond to nails which are extremely thin and peeling; such nails are usually filed using a 400 or 600 grit file and are considered extremely weak nails;

grades 4 to 6 correspond to nails of weak to medium quality which are regularly broken; such nails are usually filed using a 300 grit file and are considered weak to average nails;

grades 7 and 8 correspond to nails of good quality, which are rarely broken; such nails are usually filed using a 200 grit file and are considered good nails; and

grades 9 and 10 correspond to very thick and strong nails; such nails are usually filed using a 120 grit file and are considered excellent nails.

The four test subjects selected were between the ages of 20 to 50 and had different grades of nails. The nails of test subjects 1, 2 and 3 were treated with the nail enamel composition of Example 1 and the nails of test subject 4 were treated with the nail enamel composition of Example 2.

Test Subject 1

Test subject 1 had extremely thin and brittle nails prior to treatment with the nail enamel composition of Example 1. In particular, the nails were of grade 1 and had first been filed with a 600 grit file. At the beginning of the treatment, the subjects nails were cleansed using a nail polish remover and then, two layers of the nail enamel composition were applied to the nails. During the next four weeks, two new layers of the composition were applied each week. Prior to applying a new layer, the nails were filed and the previously-applied layer was removed using a nail remover. During the third week, considerable improvements of the nails were noticed. The nails were of grade 2, thicker and peeling was no longer observed. After five weeks of treatment, the nails improved from grade 1 to grade 3 and a 400 grit file was necessary to file the nails.

Test Subject 2

Test subject 2 had good quality nails, but the nails of both hands were of a different quality i.e. the nails of the right hand were of grade 7 and the nails of the left hand were of grade 6. The nails of both hands of test subject 2 were filed with a 200 grit file. The same procedure as described for test subject 1 was applied to the nails of test subject 2, with the exception that each week, only one layer of the nail enamel composition of Example 1 was applied instead of two, in view of the quality of the nails. During the second week, substantial improvements of the nails were noticed. The right hand nails were of grade 8 and the left hand nails were of grade 7. After five weeks of treatment, the nails of both hands were longer and all even, and an increased growth rate was observed. The nails of both hands became nails of grade 9 and a 120 grit file was now more appropriate to file the nails.

Test Subject 3

Test subject 3 had weak and short nails of grade 2, which were very brittle and of uneven length. Prior to applying the nail enamel composition of Example 1, the nails were filed with a 400 grit file. The nails of test subject 3 were treated according to the same procedure as described for test subject 1. During the third week, the nails became of grade 3, nails were longer and of even length. After five weeks, the quality of the nails increased to grade 4 for certain nails and to grade 5 for other nails, and they all needed to be filed with a coarser grit such as a 300 grit file.

Test Subject 4

Test subject 4 had grade 5 nails of average thickness prior to treatment with the nail enamel composition of Example 2. The nails were first filed with a 300 grit file prior to applying the composition. The same procedure as described for test subject 2 was applied to the nails of test subject 4, with the exception that nail enamel composition of Example 2 was applied instead of the nail enamel composition of Example 1. During the second week, the quality of the nails increased to grade 6 and, after five weeks, outstanding improvement in nail quality was observed. In particular, the nails were longer and their growth rate was greater. During the five-week treatment, the quality of the nails increased from grade 5 to grade 8 and, at the end, a 200 grit file was thus more appropriate to file them.

In view of the above-mentioned results for test subjects 1 to 4, it is apparent that the nail enamel compositions of Examples 1 and 2 improve the structure of the nails. For all the above-mentioned subjects, the treated nails appeared to be stronger and thicker, and they all showed an increased growth rate. It has been demonstrated that when the nails have a quality of grade 5 and higher, only one layer of the composition is necessary to improve the structure of these nails.

Thus, by using the above-mentioned nail enamel compositions, improvement of the structure of the nails is rapidly observed. For nails of grades 1 to 3, improvement was noticed after three weeks of treatment and, for nails of grade 4 and higher, improvement was noticed after only two weeks of treatment.

It should be understood from the experiments carried out on the above-mentioned test subjects, that the treatment using the compositions of the invention can be advantageously continued for more than five weeks and that by doing so, a user will still Improve the quality of the nails. It should also be understood that by applying the compositions of the invention more than once or twice a week, the improvement in the quality of the nails can be obtained more rapidly. For example, the treatment of test subject 1 was extended to seven weeks. In the sixth week, two new layers were applied and few days later, an additional layer was applied without removing the two layers previously applied. In the seventh week, the quality of the nails reached grade 4. The compositions of the invention can be applied to the nails by a professional nail technician or by the customer herself.

While the invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and which may be applied to the essential features hereinbefore set forth, and as follows in the scope of the appended claims.

What is claimed is:

1. A nail cosmetic composition comprising a source of calcium in powder form and a cosmetically acceptable carrier therefor, the source of calcium being selected from the group consisting of pearl, ivory, seashell and mixtures thereof.

2. The composition of claim 1, wherein said source of calcium is present in an amount of at least 0.5% by weight, based on the total weight of the composition.

3. The composition of claim 2, wherein the amount of said source of calcium is between 1.0 and 30.0% by weight.

4. The composition of claim 3, wherein the amount of said source of calcium is between 2.0 and 10.0% by weight.

5. The composition of claim 1, wherein said cosmetically acceptable carrier is present in an amount of 20 to 97% by weight, based on the total weight of the composition.

6. The composition of claim 1, wherein said cosmetically acceptable carrier is selected from the group consisting of a cream, a foam, a gel, a solution, a nail varnish composition, a nail enamel composition, a nail glue, a nail resin and a spray.

7. The composition of claim 1, wherein said source of calcium in powder form is suspended in said cosmetically acceptable carrier.

8. The composition of claim 1, wherein said source of calcium is pearl.

9. In a nail cosmetic composition comprising an active agent and a cosmetically acceptable carrier therefor, the improvement wherein said active agent is selected from the group consisting of pearl powder, ivory powder, seashell powder and mixtures thereof.

10. A nail enamel composition comprising:
a film former;
an organic solvent; and
a source of calcium in powder form, the source of calcium being selected from the group consisting of pearl, ivory, seashell and mixtures thereof.

11. The composition of claim 10, wherein said film former is present in an amount of 10 to 60% by weight, based on the total weight of the composition.

12. The composition of claim 10, wherein said film former comprises a primary film former and a secondary resin.

13. The composition of claim 12, wherein said primary film former is nitrocellulose.

14. The composition of claim 12, wherein said secondary resin is selected from the group consisting of a tosylamide epoxy resin, a tosylamide formaldehyde resin and a mixture thereof.

15. The composition of claim 10, wherein said organic solvent is present in an amount of 40 to 90% by weight, based on the total weight of the composition.

16. The composition of claim 15, wherein the amount of said organic solvent is between 50 and 75% by weight.

17. The composition of claim 10, wherein said source of calcium is present in an amount of at least 0.5% by weight, based on the total weight of the composition.

18. The composition of claim 17, wherein the amount of said source of calcium is between 1.0 and 30.0% by weight.

19. The composition of claim 18, wherein the amount of said source of calcium is between 2.0 and 10.0% by weight.

20. The composition of claim 10, wherein said composition further comprises a plasticizer.

21. The composition of claim 20, wherein said plasticizer is present in an amount of 1 to 25% by weight, based on the total weight of the composition.

22. The composition of claim 20, wherein said plasticizer is dibutyl phtalate.

23. The composition of claim 20, wherein said source of calcium in powder form is suspended in a mixture of said film former, said organic solvent and said plasticizer.

24. The composition of claim 10, wherein said source of calcium in powder form is pearl powder.

25. The composition of claim 10, further including a suspending agent.

26. The composition of claim 25, wherein said suspending agent is present in an amount of 0.10 to 15.00% by weight, based on the total weight of the composition.

27. The composition of claim 26, wherein the amount of said suspending agent is between 0.25 and 10.00% by weight.

28. The composition of claim 25, wherein said suspending agent is selected from the group consisting of attapulgite, bentonite, hectorite and mixtures thereof.

29. The composition of claim 28, wherein said suspending agent is bentonite.

30. The composition of claim 10, further including an additive selected from the group consisting of antibacterial agents, antifungal agents, botanical extracts, fragrances, moisturizers, preservatives, UV filters, proteins, stabilizers, vitamins and mixtures thereof.

31. In a nail enamel composition comprising an active agent, a film former and an organic solvent, the improvement wherein said active agent is selected from the group consisting of pearl powder, ivory powder, seashell powder and mixtures thereof.

32. The composition of claim 1, wherein said composition is capable of preventing friability of nails, improving the structure of nails, providing an increased growth rate of nails or increasing thickness of nails.

33. The composition of claim 10, wherein said composition is capable of preventing friability of nails, improving the structure of nails, providing an increased growth rate of nails or increasing thickness of nails.

34. An active agent for improving structure of nails, comprising a source of calcium in powder form, the source of calcium being selected from the group consisting of pearl, ivory, seashell and mixtures thereof.

35. The active agent of claim 34, wherein said powder is pearl powder.

36. A method of treating nails, comprising the stop of applying to said nails a nail cosmetic composition comprising a source of calcium in powder form and a cosmetically acceptable carrier therefor, the source of calcium being selected from the group consisting of pearl, ivory, seashell and mixtures thereof.

37. The method of claim 36, wherein said composition is applied to said nails at least once a week for a period of at least 2 weeks.

38. A method of treating nails, comprising the step of applying to said nails at least one layer of a nail enamel composition comprising:
a film former;
an organic solvent; and
a source of calcium in powder form, the source of calcium being selected from the group consisting of pearl, ivory, seashell and mixtures thereof.

39. The method of claim 38, wherein at least one new layer of said nail enamel composition is applied to said nails at least once a week for a period of at least 2 weeks.

40. The method of claim 39, wherein prior to apply a new layer of said nail enamel composition, the previously applied layer is removed by applying to said nails a nail polish remover.

41. The method of claim 36, wherein said method is efficient for protecting, hardening or embellishing the nails.

42. The method of claim 38, wherein said method is efficient for protecting, hardening or embellishing the nails.
